SEQUENCE LISTING (9 sequences)

SEQ ID NO: 1 - protein, 212 aa, Homo sapiens
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKSIEAVHEDIRVLSEDAIRTATEKPLGELWK

SEQ ID NO: 2 - protein, 214 aa, Mycobacterium tuberculosis
MLIAIEGVDGAGKRTLVEKLSGAFRAAGRSVATLAFPRYGQSVAADIAAEALHGEHGDLASSVYAMATLFALDRAGAVHTIQGLCRGYDVVILDRYVASNAAYSAARLHENAAGKAAAWVQRIEFARLGLPKPDWQVLLSVSAELAGERSRGRAQRDPGRARDNYERDAELQQRTGAVYAELAAQGWGGRWLVVGADVDPGRLAATLAPPDVPS

SEQ ID NO: 3 - protein, 210 aa, Plasmodium falciparum
MTDDKKKGKFIVFEGLDRSGKSTQSKLLVEYLKNNNVEVKHLYFPNRETGIGQIISKYLKMENSMSNETIHLLFSANRWEHMNEIKSLLLKGIWVVCDRYAYSGVAYSSGALNLNKTWCMNPDQGLIKPDVVFYLNVPPNYAQNRSDYGEEIYEKVETQKKIYETYKHFAHEDYWINIDATRKIEDIHNDIVKEVTKIKVEPEEFNFLWS

SEQ ID NO: 4 - protein, 210 aa, Pseudomonas aeruginosa
MTGLFVTLEGPEGAGKSTNRDYLAQRLRERGIEVQLTREPGGTPLAERIRELLLAPSDEPMAADTELLLMFAARAQHLAGVIRPALARGAVVLCDRFTDATYAYQGGGRGLPEARIAALESFVQGDLRPDLTLVFDLPVEIGLARAAARGRLDRFEQEDRRFFEAVRQTYLQRAAQAPERYQVLDAGLPLAEVQAGLDRLLPNLLERLNG

SEQ ID NO: 5 - protein, 224 aa, Candida albicans
MARGQLILIEGLDRSGKSTQASILSTKLSPSKLIKFPDRSTPIGKLINEYLTNKSFTLSDQAAHLLFSANRWELSQQIQDLLNQGYFIILDRYIYSGIAYTLAKNDFHDETISQGKNKQQLNNIDWLLSPDKGLPKPDLTLFLTLDLEEISKRKGWGDERYELQQFQAKVKQCFLEILDTNKDPTIRIVDVGGKTIDQVTTQLWEIIETNKNHELINDSIQFIT

SEQ ID NO: 6 - protein, 213 aa, Escherichia coli
MRSKYIVIEGLEGAGKTTARNVVVETLEQLGIRDMVFTREPGGTQLAEKLRSLVLDIKSVGDEVITDKAEVLMFYAARVQLVETVIKPALANGTWVIGDRHDLSTQAYQGGGRGIDQHMLATLRDAVLGDFRPDLTLYLDVTPEVGLKRARARGELDRIEQESFDFFNRTRARYLELAAQDKSIHTIDATQPLEAVMDAIRTTVTHWVKELDS

SEQ ID NO: 7 - protein, 345 aa, Trypanosoma brucei brucei
MVAVNRKIVVFTDVEDKQVLEVIREVAGDAETIQLEKSFAYEFMWRGKHTPYRVGTPMCFITEKTVEDADVVFAVYSKRVPVLSLTTGGETTNLLETISSLGIECKQNTSETWADAVRAFLPNNNSESAVIVFEGGDGAGKATQTEYMRKRLDKEGKAHASLDFPSDKHRYGKLIRKVLSGERGALHDLDPKLFSLLFSLNRFDTLPELRYWLTHGKKILLDRYYTSNFGHQASKLEESERSDFIRHLEQLELEWLKLPPPDVALYLDLPPAAALVAMQRDEKRESLDIHETAGLSYKENVRQTYVWCCQNQRNWVQIECCNDEGERYSREKVHEMIYNAVAKHI

SEQ ID NO: 8 - protein, 194 aa, Homo sapiens
MEEKLKKTKIIFVVGGPGSGKGTQCEKIVQKYGYTHLSTGDLLRSEVSSGSARGKKLSEIMEKGQLVPLETVLDMLRDAMVAKVNTSKGFLIDGYPREVQQGEEFERRIGQPTLLLYVDAGPETMTQRLLKRGETSGRVDDNEETIKKRLETYYKATEPVIAFYEKRGIVRKVNAEGSVDSVFSQVCTHLDALK

SEQ ID NO: 9 - protein, 222 aa, Homo sapiens
MHHHHHHGSTSAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKSIEAVHEDIRVLSEDAIRTATEKPLGELWK

(19) United States
(12) Patent Application Publication
(10) Pub. No.: US 2017/0306388 A1
(43) Pub. Date: Oct. 26, 2017

(54) Method for the Identification of Inhibitors and Activators of Thymidylate Kinases

(71) Applicant: Carnegie Mellon University, Pittsburgh, PA (US)

(72) Inventors: Gordon Steven Rule, Pittsburgh, PA (US); Kaustubh Sinha, Pittsburgh, PA (US)

(21) Appl. No.: 15/493,509

(22) Filed: Apr. 21, 2017

Related U.S. Application Data
(60) Provisional application No. 62/391,200, filed on Apr. 22, 2016.

(51) Int. Cl. C12Q 1/48; C12N 9/12; G01N 24/08

(52) U.S. Cl. CPC C12Q 1/485; C12Y 207/04009; G01N 24/088; C12N 9/1229; C12Y 207/04003; G01N 2333/91235

(57) ABSTRACT

Provided herein is a method of producing large amounts of the enzyme thymidylate kinase from multiple species for drug discovery purposes. Also provided herein are NMR methods of monitoring a reaction involving a thymidine kinase.

### CROSS REFERENCE TO RELATED APPLICATIONS

This application claims the benefit of U.S. Provisional Patent Application No. 62/391,200, filed Apr. 22, 2016, which is incorporated herein by reference in its entirety.

A method for the identification of inhibitors and activators of thymidylate kinases is provided, for example for drug discovery. Methods and compositions for the production of thymidylate kinases are provided.

Thymidylate kinases (TMKs) play a central role in nucleotide metabolism (it is the only enzyme that converts dTMP (deoxythymidine monophosphate) to dTDP (deoxythymidine diphosphate)). It has been recognized as a suitable target for lead compound development and a number of groups have developed inhibitors against TMKs from gram positive bacteria, Plasmodium falciparum, Mycobacterium tuberculosis, and Pseudomonas aeruginosa. TMKs are also involved in the activation of pro-drugs. In the case of antivirals against herpes simplex and varicella-zoster, the viral TMK is responsible for conversion of the pro-drug to the active triphosphorylated form.

huTMK is also required for rapidly growing cancerous cells; hence its inhibition may complement other chemotherapeutic approaches. In summary, TMKs are an attractive therapeutic target for a wide range of diseases, ranging from bacterial, parasitic, and fungal infections to cancer.

There are currently three reported methods to assay TMK activity. The first method uses isotope labeled dTMP as substrate and utilizes the separation of dTMP and dTDP by thin-layer chromatography. The next method is a coupled enzymatic assay which involves the use of pyruvate kinase and lactate dehydrogenase to couple the production of ADP (adenosine diphosphate) to the oxidation of NADH to NAD+ which can be monitored spectrophotometrically at 340 nm. The third protocol involves the use of luciferin/luciferase to measure the concentration of unreacted ATP (adenosine triphosphate), or the production of ADP (adenosine diphosphate) in the TMK reaction mixture. All of these assays have inherent problems, ranging from the use of radioactivity to the potential inhibition of the coupling enzymes by the lead compound in high-throughput screening.

TMKs are an attractive target for therapeutics for a wide range of diseases, ranging from bacterial, parasitic, and fungal infections to cancer, but effective methods of identifying both inhibitors and activators of the enzyme are lacking.

### SUMMARY

Provided herein are methods of identifying activators and inhibitors of TMK activity. Provided herein also are methods of producing large amounts of TMK enzyme.

According to one aspect, a method of preparing an enzyme is provided. The method comprises: culturing a cell comprising an exogenous gene for expressing an enzyme in cell culture medium under conditions suitable for expression of the exogenous gene in the cell; and isolating the enzyme from the cell by lysing the cell in a lysis solution to produce a lysate, binding the enzyme in the lysate to a selectivity component specific to the enzyme that is optionally bound to a surface, and separating the bound enzyme from the lysate, wherein the enzyme is bound to the binding reagent in the presence of a substrate of the enzyme.

According to another aspect, a method of monitoring an enzymatic reaction of a nucleoside phosphate kinase is provided. The method comprises: conducting a reaction of the nucleoside phosphate kinase in a reaction mixture comprising a pyrimidine substrate of the nucleoside phosphate kinase having a pyrimidine H6 proton or a purine substrate of the nucleoside phosphate kinase having a purine H8 proton; and conducting a nuclear magnetic resonance (NMR) assay on the reaction mixture during the course of the reaction (that is, at any time frame in which the reaction is proceeding) to identify or determine the presence of a shift in a peak of the spectrum corresponding to the pyrimidine H6 proton or purine H8 proton, wherein a shift in the peak corresponding to the pyrimidine H6 proton or purine H8 proton indicates enzymatic conversion of the substrate to the product of the enzymatic reaction.

According to another aspect, a method determining the effect of a composition, compositions, or library of compositions on an enzymatic reaction of a nucleoside phosphate kinase is provided. The method comprises: conducting a reaction of the nucleoside phosphate kinase in a reaction mixture comprising a pyrimidine substrate of the nucleoside phosphate kinase having a pyrimidine H6 proton or a purine substrate of the nucleoside phosphate kinase having a purine H8 proton and a sample composition; conducting a nuclear magnetic resonance (NMR) assay on the reaction mixture during the course of the reaction (that is, at any time frame in which the reaction is proceeding) to identify or determine the presence of a shift in a peak of the spectrum corresponding to the pyrimidine H6 proton or the purine H8 proton, wherein a shift in the peak corresponding to the pyrimidine H6 proton or the purine H8 proton indicates enzymatic conversion of the substrate to the product of the enzymatic reaction; comparing, the rate and/or extent of enzymatic conversion of the substrate to the product of the enzymatic reaction to a rate and/or extent of enzymatic conversion of the substrate to the product of the enzymatic reaction in a reaction mixture either excluding the sample composition or having a different amount of the sample composition; and identifying sample compositions that affect the rate and/or extent of enzymatic conversion of the substrate to the product of the enzymatic reaction.

### BRIEF DESCRIPTION OF THE DRAWINGS

FIGS. 1A-1G provide exemplary protein sequences of TMK proteins of: Homo sapiens, isoform 1 (SEQ ID NO: 1, FIG. 1A); Mycobacterium tuberculosis (SEQ ID NO: 2, FIG. 1B); Plasmodium falciparum 3D7, putative (SEQ ID NO: 3, FIG. 1C); Pseudomonas aeruginosa, dTMP kinase (SEQ ID NO: 4, FIG. 1D); Candida albicans SC5314 bifunctional thymidylate/uridylate kinase (SEQ ID NO: 5, FIG. 1E); Escherichia coli (SEQ ID NO: 6, FIG. 1F); Trypanosoma brucei brucei TREU927 (SEQ ID NO: 7, FIG. 1G). FIG. 1H provides an exemplary sequence of an adenylate kinase enzyme FAK1 adenylate kinase 1 [Homo sapiens (human)] NCBI Gene ID: 203, GenBank Ref. No: NP_000467; SEQ ID NO: 8)

FIG. 2 depicts a structure of pAZT (azidothymidine monophosphate).

FIG. 3 depicts structures of common nucleobases: adenine (A), guanine (G), cytosine (C), thymine (T), and uracil (U), where [symbol] indicates the bond connecting the nucleobase to a ribose or deoxyribose moiety of a composition as described herein. The H6 protons are depicted for the pyrimidines, and the H8 protons are depicted for the purines, with C6 and C8 being labeled for the pyrimidines and purines, respectively.

FIG. 4 illustrates the NMR Based Continuous Enzyme Assay for TMK. The H6 proton resonance for TMP is at 8.02 ppm. Addition of TMK generates TMP+TDP, H6 resonance of TDP is at 7.94.

### DETAILED DESCRIPTION

Other than in the operating examples, or where otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical values, however, inherently contain certain errors necessarily resulting from the standard deviation found in their respective testing measurements. Furthermore, when numerical ranges of varying scope are set forth herein, it is contemplated that any combination of these values inclusive of the recited values may be used.

Also, it should be understood that any numerical range recited herein is intended to include all sub-ranges subsumed therein. For example, a range of “1 to 10” is intended to include all sub-ranges between and including the recited minimum value of 1 and the recited maximum value of 10, that is, having a minimum value equal to or greater than 1 and a maximum value of equal to or less than 10.

As used herein “a” and “an” refer to one or more.

As used herein, the term “comprising” is open-ended and may be synonymous with “including”, “containing”, or “characterized by”.

By “expression” or “gene expression,” it is meant the overall flow of information from a gene, operon or equivalent structure. A gene is, without limitation, a functional genetic unit for producing a gene product, such as an RNA or a protein in a cell, or other expression system. A gene is encoded on a nucleic acid and comprises: a) a transcriptional control sequence, such as a promoter, operator, or other cis-acting elements, such as a Pribnow box or TATA box, or transcriptional response elements (TREs) and/or enhancers; and b) an expressed sequence that typically encodes a protein (referred to as an open-reading frame or ORF) or functional/structural RNA, and a polyadenylation sequence. The gene produces a gene product that is typically a protein that is optionally post-translationally modified, or a functional/structural RNA. A “fusion protein” is a protein produced from an ORF that contains sequences from two or more proteins, including proteins comprising affinity tags, as are described herein.

By “expression of a gene under transcriptional control of,” or alternately “subject to control by,” a designated sequence such as a promotor, operator, TRE, or other transcription control element, it is meant gene expression from a gene containing the designated sequence operably linked (functionally attached, typically in cis) to the gene. A gene that is “under transcriptional control” of a promoter, operator, TRE, or other transcription control element, is a gene that is transcribed at detectably different levels in the presence of a suitable RNA polymerase and any necessary transcription factors or other compositions necessary for transcription of the gene, and in the context of the present disclosure, produces an enzyme, such as a TMK protein. A “gene for expression of” a stated gene product is a gene capable of expressing that stated gene product when placed in a suitable environment—that is, for example, when transformed, transfected, transduced, etc. into a cell, and subjected to suitable conditions for expression. In the case of a constitutive promoter “suitable conditions” means that the gene typically need only be introduced into a host cell. In the case of an inducible promoter or operator, “suitable conditions” means when factors that regulate transcription, such as RNA polymerase, sigma factors or and/or other DNA-binding proteins or transcription-promoting factors, are present or absent in amounts effective to cause expression of the gene.

Production of useful nucleic acid constructs, such as genes for production of a given enzyme, such as TMK, for example human TMK, is routine, in that broadly-known molecular cloning procedures are routine, and recombinant constructs useful for constructing a gene for expression of an ORF, such as a TMK ORF, in prokaryotes (e.g. E. coli), or eukaryotes (e.g., yeast, insect or mammalian cells) are broadly-available commercially (e.g., from Addgene of Cambridge, Mass., or ThermoFisher Scientific, Inc., including the T7 Expression System as described herein), and are abundantly described in the literature. As such, preparing a construct for expression of an enzyme such as a TMK, such as huTMK is considered routine.

An “exogenous” gene, operon, or nucleic acid is a gene, operon, or nucleic acid that originates outside of the organism. A recombinant genetic construct that is not part of the normal genome of the organism, and is introduced into the cell, such as the T7-expressed TMK constructs described herein, introduced into the E. coli host cell are considered to be exogenous.

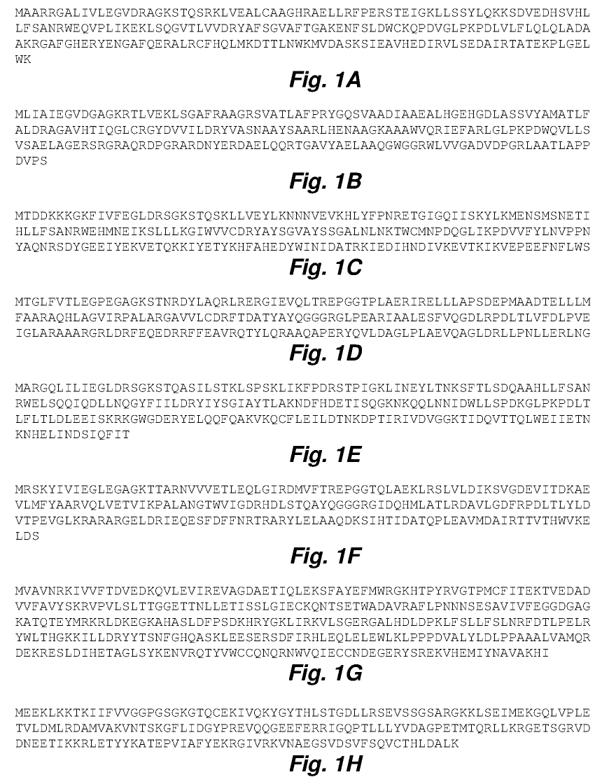

Thymidylate kinase (TMK) is a ubiquitous enzyme that catalyzes the production to thymidine 5′-diphosphate from thymidine 5′ monophosphate in the presence of adenosine triphosphate and Mg²⁺. FIGS. 1A-1G provide exemplary protein sequences of TMK proteins of: Homo sapiens, isoform 1 (GenBank Ref. No. NP_036277.2, SEQ ID NO: 1, FIG. 1A); Mycobacterium tuberculosis (GenBank Ref. No. WP_057369853.1, SEQ ID NO: 2, FIG. 1B); Plasmodium falciparum 3D7, putative (GenBank Ref. No. XP_001350897.1, SEQ ID NO: 3, FIG. 1C); Pseudomonas aeruginosa, dTMP kinase (GenBank Ref. No. WP_050160013.1, SEQ ID NO: 4, FIG. 1D); Candida albicans SC5314 bifunctional thymidylate/uridylate kinase (GenBank Ref. No. XP_713007.1, SEQ ID NO: 5, FIG. 1E); Escherichia coli (GenBank Ref. No. WP_001748292.1, SEQ ID NO: 6, FIG. 1F); Trypanosoma brucei brucei TREU927 (GenBank Ref. No. XP_847222.1, SEQ ID NO: 7, FIG. 1G).

As used herein in certain aspects the enzyme is bound to the binding reagent in a molar excess of the substrate, meaning the relative number of molecules of the substrate is greater than the overall number of substrate-binding sites available in the enzyme polypeptides in the solution containing the enzyme, binding reagent, and substrate.

A “selectivity component” is a molecular recognition unit, such as a polypeptide, polypeptide analog, glycoprotein, nucleic acid, nucleic acid analog, polysaccharide, ligand, or any other molecule or composition that recognizes and binds specifically to a target analyte, e.g., a biomolecule, such as an RNA, DNA, protein, glycoprotein, polysaccharide, lipid, or other cellular constituent, or combinations thereof with limited or no cross-reactivity to other compounds or compositions in a given system, that is, they are binding reagents that bind specifically compounds, molecules or compositions to the substantial exclusion of others in a given reaction, such as in the purification of a recombinant protein from a cell lysate as described herein.

In the context of the production of recombinant proteins, e.g., by affinity purification, the selectivity component is specific enough in its binding to permit isolation of the target protein with non-interfering cross-reactivity with non-target analytes and/or non-specific binding.

In certain aspects, the selectivity component is an antibody or an antibody fragment. For example, activators may be monoclonal antibodies, or derivatives or analogs thereof, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent activators including without limitation: monospecific or bispecific antibodies, such as disulfide stabilized Fv fragments, scFv tandems ((scFv)2 fragments), diabodies, tribodies or tetrabodies, which typically are covalently linked or otherwise stabilized (i.e., leucine zipper or helix stabilized) scFv fragments; receptor molecules which naturally interact with a desired target molecule.

In one aspect, the selectivity component is an antibody. Preparation of antibodies may be accomplished by any number of well-known methods for generating monoclonal antibodies. These methods typically include the step of immunization of animals, typically mice; with a desired immunogen (e.g., a desired target molecule-or fragment thereof). Once the mice have been immunized, and preferably boosted one or more times with the desired immunogen(s), monoclonal antibody-producing hybridomas may be prepared and screened according to well-known methods (see, for example, Owen, J. A. et al., KUBY IMMUNOLOGY, Seventh Edition, pp. 654-656, W.H. Freeman & Co. (2013), for a general overview of monoclonal antibody production). Production of antibodies and other binding reagents have become extremely robust. In vitro methods that combine antibody recognition and phage display techniques allow one to amplify and select antibodies or other binding reagents with very specific binding capabilities. These methods typically are much less cumbersome than preparation of hybridomas by traditional monoclonal antibody preparation methods. Binding epitopes may range in size from small organic compounds such as bromo uridine and phosphotyrosine to oligopeptides on the order of 7-9 amino acids in length.

In another aspect, the selectivity component is an antibody fragment. Selection and preparation of antibody fragments may be accomplished by any number of well-known methods. Phage display, bacterial display, yeast display, mRNA display and ribosomal display methodologies may be utilized to identify and clone desired antibody fragment activators that are specific for a desired target molecule, including, for example, Fab fragments, FVs with an engineered intermolecular disulfide bond to stabilize the VH-VL pair, scFvs, or diabody fragments. Production of scFv antibody fragments using display methods, including phage, bacterial, yeast, ribosomal and mRNA display methods can be employed to produce the activator and/or selectivity component, as described herein. In still other aspects, the selectivity component may be an aptamer, also known as a nucleic acid ligand. Aptamers are oligonucleotides that are selected to bind specifically to a desired molecular structure. Aptamers typically are the products of an affinity selection process similar to the affinity selection of phage display (also known as in vitro molecular evolution).

The selectivity component binds the enzyme to be isolated. In certain aspects, the enzyme is a fusion protein that contains a tag or handle which facilitates its isolation, immobilization, identification, or detection and/or which increases its solubility. In various aspects, the tag may be a polypeptide, a polynucleotide, a carbohydrate, a polymer, or a chemical moiety and combinations or variants thereof. In certain embodiments, exemplary chemical handles, include, for example, glutathione S-transferase (GST), protein A, protein G, calmodulin-binding peptide, thioredoxin, maltose binding protein, HA, myc, poly arginine, poly His, poly His-Asp or FLAG tags. Additional exemplary tags include polypeptides that alter protein localization in vivo, such as signal peptides, type III secretion system-targeting peptides, transcytosis domains, nuclear localization signals, etc. In one aspect, the tag is removable from the enzyme, meaning it can be cleaved from the enzyme after affinity purification, for example, by the TEV cleavage site. The tag or handle can be incorporated into the protein by including the sequence thereof in-frame in the ORF of the enzyme, e.g. at the N- or C-terminal end, for example as described above for huTMK cloning, and as is well understood in the field of production of recombinant proteins.

NMR devices are broadly-available and include networkable, benchtop high-resolution NMR devices, for example from ThermoFisher (picoSpin™) and Oxford Instruments (PULSAR™), as well as more conventional devices. NMR devices can be connected to suitable computer systems for monitoring changes in the NMR spectrum as described herein. A computer process can be utilized to monitor any changes in the amounts of a reaction product. A computer comprises a processor, a user interface, and a non-transitory computer readable storage medium having stored thereon a computer program comprising instructions, which, when executed by the computer processor, cause the computer to produce an output. The processor, user interface, and non-transitory computer readable storage medium do not necessarily have to be located in the same physical location, and thus, the computer can take on many physical forms, including a desktop, laptop, smartphone, terminal, networked computer system, server, etc. The computer may utilize, for example and without limitation, any suitable and compatible hardware, BIOS, operating system, programming language, peripherals, displays, printers, speakers, communication hardware and protocols (e.g., wired or wireless). In one example, the computer identifies and provides output, e.g., through a display or printer, indicating progression of an enzymatic reaction, for example and without limitation, by differencing two data sets. Differencing refers to a process by which a computer takes two data sets and identifies differences between the two data sets, such that the computer process produces an output that identifies and/or quantifies the difference between the two data sets. For example, in the context of the NMR-based assay described herein, the area, height, and/or other qualities of peaks corresponding to the pyrimidine H6 proton or the purine H8 proton at two time points during an enzymatic reaction can be compared by differencing and the rate or extent of a nucleoside phosphate kinase enzymatic reaction can be calculated and output by the computer.

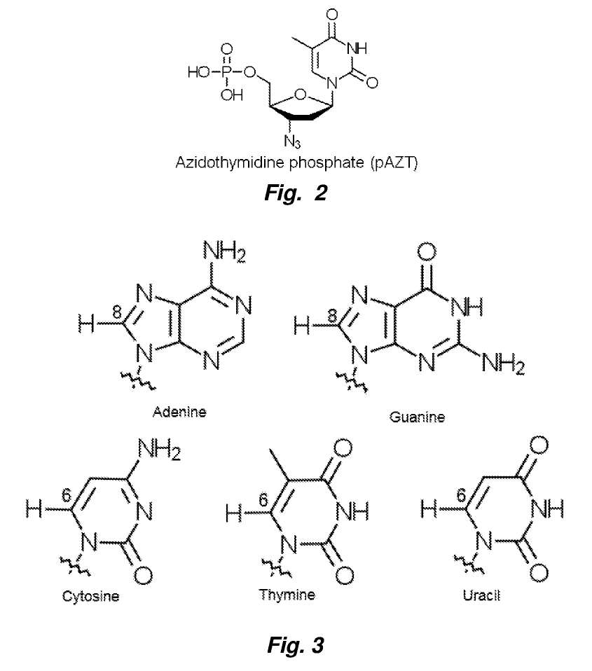

According to one aspect of the invention, a method of preparing a TMK enzyme is provided. The method produces large amounts of the thymidylate kinase, e.g., for the NMR methods described herein, and more generally for drug discovery purposes. The method comprises: culturing a cell comprising an exogenous gene for expressing a TMK enzyme in cell culture medium under conditions suitable for expression of the exogenous gene in the cell; and isolating the TMK enzyme from the cell by lysing the cell in a lysis solution to produce a lysate, binding the TMK enzyme in the lysate to a selectivity component specific to the enzyme that is bound to a surface (e.g., a tube, flask, cover-slip, plastic article, beads, fluorescent- or quantum dot-labeled beads, magnetic beads, nanoparticles, etc.), and separating the bound enzyme from the lysate, wherein the enzyme is bound to the binding reagent in the presence of a substrate of the enzyme. As used herein, a “substrate” of an enzyme is a molecule upon which the enzyme acts in catalyzing a chemical reaction to modify the substrate. In the example of a TMK enzyme, a suitable substrate is TMP, though other substrates, such as pAZT (FIG. 2) or GMP (guanosine monophosphate) can bind to the TMK active site. The substrate can be eluted/dissociated from the isolated enzyme using non-denaturing amounts of a protein denaturant, such as from 0.5 M to 3 M urea, or equivalent amounts of guanidinium HCl. A non-denaturing amount of a protein denaturant can temporarily disrupt the protein structure so that the substrate is released, but permits recovery of the activity of the enzyme upon dilution of, or removal of the denaturant.

In one aspect, the method uses heterologous expression in E. coli to obtain high amounts of the enzyme. Production of these enzymes in E. coli allows cost-effective isotopic labeling. For example, the enzyme can be from the following species: human; Mycobacterium tuberculosis, Plasmodium falciparum, Pseudomonas aeruginosa, Candida albicans, Escherichia coli, or Trypanosoma brucei brucei. Thus the present invention provides the appropriate materials for drug screening for anti-malarial, anti-fungal, anti-HIV, and anti-cancer drugs.

According to one aspect of the invention, a method is provided for monitoring an enzymatic reaction of a nucleoside phosphate kinase. A nucleoside phosphate kinase, including nucleoside monophosphate kinases and nucleoside diphosphate kinases, is an enzyme that catalyzes the transfer of a phosphate group from a phosphate-donating molecule to a purinyl or pyrimidinyl monophosphate or diphosphate nucleoside, and includes a TMK enzyme as described herein, as well as, without limitation, adenylate kinase (see, e.g., FIG. 1H, AK1 adenylate kinase 1 [Homo sapiens (human)] NCBI Gene ID: 203, GenBank Ref. No: NP 000467; and which is found in organisms including bacteria, fungi, and animals, including, without limitation: Plasmodium, Candida, Mycobacterium, Trypanosoma, and E. coli organisms described herein), and other nucleoside phosphate kinases, such as, without limitation, cytidine monophosphate kinase, guanylate monophosphate kinase, uridine monophosphate kinase, and nucleoside diphosphate kinase (e.g. NME1 and NME2 in humans), all of which are broadly known for a variety of species and pathogens. As an example, a substrate of adenylate kinase is adenosine monophosphate (AMP), which is converted by the enzyme to the ADP product. Various isoforms of adenylate kinase are broadly-known, and protein and nucleotide sequences for those isoforms across a variety of species are broadly-known and published. Substrates for nucleoside diphosphate kinases are, generally, nucleoside diphosphates. The method comprises conducting a reaction of the nucleoside monophosphate nucleoside phosphate kinase in a reaction mixture comprising a pyrimidine substrate of the nucleoside phosphate kinase having a pyrimidine H6 proton or a purine substrate of the nucleoside phosphate kinase having a purine H8 proton; and conducting a nuclear magnetic resonance (NMR) assay on the reaction mixture during the course of the reaction to identify or determine the presence of a shift in a peak of the spectrum corresponding to the pyrimidine H6 proton or purine H8 proton, wherein a shift in the peak corresponding to the pyrimidine H6 proton or purine H8 proton indicates enzymatic conversion of the substrate to the product of the enzymatic reaction. The “course of the reaction” refers to a time frame in which the reaction proceeds. The assay is a simple, one-step assay that does not require use or radiolabeled compounds or enzymatic coupling. Further, the method allows for the near-continuous measurement of product production, while current methods used for high-throughput screening are quench-stop type assays. The assay can be readily partially or fully automated by one of ordinary skill in the field of the invention using suitable robotics, fluidics, and control systems, such as computer control systems, as are broadly known and available in the art.

According to another aspect of the invention, a method determining the effect of a composition on an enzymatic reaction of a nucleoside phosphate kinase, such as a TMK, is provided. The method comprises: conducting a reaction of the nucleoside phosphate kinase in a reaction mixture comprising a pyrimidine substrate of the nucleoside phosphate kinase having a pyrimidine H6 proton or a purine substrate of the nucleoside phosphate kinase having a purine H8 proton and a sample composition; conducting a nuclear magnetic resonance (NMR) assay on the reaction mixture during the course of the reaction to identify or determine the presence of a shift in a peak of the spectrum corresponding to the pyrimidine H6 proton or the purine H8 proton, wherein a shift in the peak corresponding to the pyrimidine H6 proton or the purine H8 proton indicates enzymatic conversion of the substrate to the product of the enzymatic reaction; comparing, the rate and/or extent of enzymatic conversion of the substrate to the product of the enzymatic reaction to a rate and/or extent of enzymatic conversion of the substrate to the product of the enzymatic reaction in a reaction mixture either excluding the sample composition or having a different amount of the sample composition; and identifying sample compositions that affect the rate and/or extent of enzymatic conversion of the substrate to the product of the enzymatic reaction. The comparing and identifying may be performed by a computer that produces an output that identifies sample compositions that affect the rate and/or extent of enzymatic conversion of the substrate to the product of the enzymatic reaction. A “sample composition” is a molecule, compound, or composition that is to be tested for its effect on the nucleoside phosphate kinase activity, e.g., in a drug discovery method. Thus, the sample composition includes at least a compound, such as a compound disclosed herein, which is tested using an NMR screening process for its effect on the activity of a nucleoside phosphate kinase, such as a TMK enzyme as described herein. Types of drugs that can be screened by the present invention include antibacterial, antimalarial, antifungal, and certain classes of anti-HIV drugs. In addition, compounds that inhibit the human enzyme have potential anticancer activity.

### Example 1—Preparation of Tb TMK (Mycobacterium tuberculosis Thymidylate Kinase)

A first step towards a detailed study of TbTMK is the production of mg quantities of the protein. There was great success in employing a combination of codon-optimized gene sequence and optimized growth conditions to achieve high protein yields of huTMK and PfTMK (P. falciparum thymidylate kinase) [60-250 mg/L] in D20 containing media with E. coli. A similar approach is used to express TbTMK in E. coli. The gene is designed to include a His-Tag at the N-terminus followed by a flexible linker and a unique BamHI site to insert an optional TEV cleavage site. A variety of growth conditions—temperature (22° C., 30° C. and 37° C.), IPTG concentrations [0.1 mM to 1 mM] and induction times are sampled to arrive at the optimal expression protocol. The activity of the enzyme with TMP and AZTMP (pAZT) as the phosphate acceptors is studied. Also, amide and methyl spectra are gathered of the unliganded and ligand-bound (TMP+/−ADP; AZTMP+/−ADP) protein. Kinetic and NMR spectroscopic data enables the process of screening drug libraries for TbTMK inhibitors as described above. Efforts to crystallize the protein are also made in parallel and positive results help the process of inhibitor development.

### Example 2—Expression and Purification of Human Thymidylate Kinase in E. coli

A synthetic gene codon-optimized for expression in E. coli was obtained from DNA 2.0 and cloned into the pet22b(+) vector using the NdeI and XhoI sites on the plasmid. The protein was expressed in C3013 cells (New England Biolabs) and has the following amino acid sequence:

(SEQ ID NO: 9)
MHHHHHHGSTSAARRGALIVLEGVDRAGKSTQSRKLVEALCAAGHRAELL
RFPERSTEIGKLLSSYLQKKSDVEDHSVHLLFSANRWEQVPLIKEKLSQG
VTLVVDRYAFSGVAFTGAKENFSLDWCKQPDVGLPKPDLVLFLQLQLADA
AKRGAFGHERYENGAFQERALRCFHQLMKDTTLNWKMVDASKSIEAVHED
IRVLSEDAIRTATEKPLGELWK

The cells were grown in LB media containing 100 mg/mL ampicillin at 30° C. to an A₆₀₀ of 0.8 and induced with 1 mM isopropyl-β-D-thiogalactoside (IPTG) for 4 hours. After harvesting, the cells were stored at −80° C. SDS-PAGE gel electrophoresis was used to estimate the yield of the protein and it was estimated to be 100-200 mg of protein per liter of culture.

Initial attempts to purify the protein involved re-suspending the cell pellets in buffer A (50 mM potassium phosphate, 300 mM sodium chloride, 10 mM imidazole, pH 7.4) followed by cell lysis by sonication. The lysate was centrifuged at 20,000 g for 40 minutes at 4° C. Appropriate amount of HisPur™ Cobalt Resin (Thermo Scientific) was then added to the supernatant and mixed on end-over-end rotator for 1 hour. This supernatant-resin mix was then poured into a gravity chromatography column to separate the resin from the flow-through. The resin was then washed with 3 column volumes of buffer A. The elution step was then carried out using buffer B (50 mM sodium phosphate, 300 mM sodium chloride, 150 mM imidazole, pH 7.4).

However, on quantification, it was seen that the eluent did not contain most of the protein. Almost all the protein was found to have precipitated. Different buffer conditions (pH, addition of detergents) were used to try and maintain the protein in the soluble fraction but these did not work. Finally, 50 μM of dTMP (a substrate for the enzyme with μM dissociation constant (K_{d}), that is strong binding) was added to the lysis buffer and this helped stabilize the protein and keep it in the soluble fraction. We now consistently achieve yields of −100 mg of protein per liter of culture.

### Example 3—Activity Assay Using Proton NMR

Inhibition Assays for TMK:

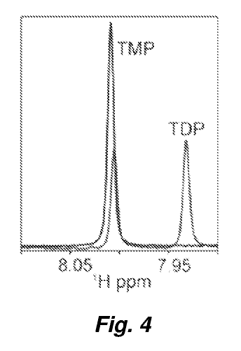

Three different methods to assay for TMK activity have been reported in the literature. The first method uses isotope labeled [¹⁴C] dTMP as substrate and utilizes the separation of dTMP and dTDP by thin-layer chromatography (TLC). The next method is a coupled enzymatic assay which involves the use of pyruvate kinase and lactate dehydrogenase to couple the production of ADP (a product of TMK activity) to the oxidation of NADH to NAD⁺ which can be monitored spectrophotometrically at 340 nm. The third protocol involves the use of luciferin/luciferase to measure the concentration of unreacted ATP, or the amount to ADP in the TMK reaction mixture. All of these assays have inherent problems, ranging from the use of radioactivity to the potential inhibition of the coupling enzymes. Consequently, a simple one step assay to monitor the conversion of TMP to TDP was developed. The assay relies on the difference in the pyrimidine H6 proton chemical shifts of TMP and TDP (see FIG. 4). The assay does not require coupling to any other enzyme, nor does it require the use of isotopically labeled compounds. The new assay of the present invention also allows for the near-continuous measurement of product production, while current high-throughput assays are quench-stop type assays.

One-dimensional proton (¹H) NMR was used to monitor the activity of CaTMPK. The assay depends on the change in the chemical shift of the H6 proton (dTMP, AZTMP) or the H8 proton (dGMP) of the nucleobases upon the addition of the second phosphate group. The assay reaction included 50 mM Tris-HCl (pH 7.4), 50 mM KCl, 10 mM MgCl₂, 1 mM ATP as the phosphate donor, 1 mM phosphate acceptor (dTMP, AZTMP or dGMP) and 10% D20. The reaction was initiated by the addition of CaTMPK to the reaction mix. A series of ¹H NMR spectra were acquired and the area (or height) of the proton line corresponding to the monophosphate forms of the acceptor molecule was plotted against time to calculate the initial rates of catalysis for each of the three substrates.

Coupled Enzyme Assay:

The NADH coupled enzyme activity assay (Blondin, C., et al. (1994). Improved spectrophotometric assay of nucleoside monophosphate kinase activity using the pyruvate kinase/lactate dehydrogenase coupling system. Anal. Biochem 220, 219-221) is a well established assay to monitor the enzyme activity of thymidylate kinases. The assay was used to measure the activity of CaTMPK with the different phosphate acceptors (dTMP, AZTMP or dGMP). The reaction mixture consisted of 50 mM Tris-HCl (pH 7.4), 50 mM KCl, 10 mM MgCl₂, 0.5 mM phosphoenolpyruvate, 0.15 mM NADH, 5 units/mL each of pyruvate kinase and lactate dehydrogenase. The reaction was initiated by adding the enzyme and the reaction rate was measured by monitoring the decrease of NADH concentration at 340 nm.

The table below compares the specific activity of CaTMPK with the 3 substrates using the NMR and the coupled activity assays, validating the NMR based assay

TABLE 1
k_{cat} of CaTMPK with dTMP, AZTMP and dGMP derived
using the NMR and coupled enzyme method.

| | | kcat, coupled enzyme assay |
|---|---|---|
| | kcat, nmr assay (min⁻¹) | (min⁻¹) |
| dTMP | 500 | 411 |
| AZTMP | 32 | 18.3 |
| dGMP | 0.37 | 0.5 |

The following numbered clauses provide examples of various aspects of the present invention.
1. A method of preparing an enzyme, comprising:
   a. culturing a cell comprising an exogenous gene for expressing an enzyme in cell culture medium under conditions suitable for expression of the exogenous gene in the cell;
   b. isolating the enzyme from the cell by lysing the cell in a lysis solution to produce a lysate, binding the enzyme in the lysate to a selectivity component specific to the enzyme that is optionally bound to a surface, and separating the bound enzyme from the lysate, wherein the enzyme is bound to the binding reagent in the presence of a substrate of the enzyme.
2. The method of clause 1, wherein the lysis solution comprises the substrate.
3. The method of clause 1, wherein the enzyme is bound to the selectivity component in a molar excess of the substrate.
4. The method of clause 1, further comprising, after isolating the enzyme from the cell, dissociating the substrate from the enzyme.
5. The method of clause 2, wherein the substrate is dissociated from the enzyme by contacting the enzyme with non-denaturing amounts of a protein denaturant.
6. The method of clause 3, wherein the enzyme is contacted with from 0.5 M to 3 M urea, or equivalent amounts of guanidinium HCl, to dissociate the substrate from the enzyme.
7. The method of clause 5, wherein the enzyme is a Homo sapiens, Mycobacterium tuberculosis, Plasmodium falciparum, Pseudomonas aeruginosa, Candida albicans, Escherichia coli, or Trypanosoma brucei brucei thymidylate kinase.
8. A method of monitoring an enzymatic reaction of a nucleoside phosphate kinase, comprising:
   a. conducting a reaction of the nucleoside phosphate kinase in a reaction mixture comprising a pyrimidine substrate of the nucleoside phosphate kinase having a pyrimidine H6 proton or a purine substrate of the nucleoside phosphate kinase having a purine H8 proton; and
   b. conducting a nuclear magnetic resonance (NMR) assay on the reaction mixture during the course of the reaction to identify or determine the presence of a shift in a peak of the spectrum corresponding to the pyrimidine H6 proton or purine H8 proton,
      wherein a shift in the peak corresponding to the pyrimidine H6 proton or purine H8 proton indicates enzymatic conversion of the substrate to the product of the enzymatic reaction.
9. The method of clause 8, wherein the nucleoside phosphate kinase is a thymidylate kinase and the shift in the peak of a thymine H6 proton is measured.
10. The method of either clause 8 or 9, comprising determining the rate of the enzymatic conversion of the substrate to the product of the enzymatic reaction or extent of the enzymatic conversion of the substrate to the product of the enzymatic reaction by comparing the spectrum peak or peaks corresponding to the pyrimidine H6 proton or purine H8 proton at two time points during which the enzymatic reaction is conducted.
11. The method of any one of clauses 8-10, wherein the nucleoside phosphate kinase is an adenylate kinase.
12. A method determining the effect of a composition on an enzymatic reaction of a nucleoside phosphate kinase, comprising:
   a. conducting a reaction of the nucleoside phosphate kinase in a reaction mixture comprising a pyrimidine substrate of the nucleoside phosphate kinase having a pyrimidine H6 proton or a purine substrate of the nucleoside phosphate kinase having a purine H8 proton and a sample composition;
   b. conducting a nuclear magnetic resonance (NMR) assay on the reaction mixture during the course of the reaction to identify or determine the presence of a shift in a peak of the spectrum corresponding to the pyrimidine H6 proton or the purine H8 proton, wherein a shift in the peak corresponding to the pyrimidine H6 proton or the purine H8 proton indicates enzymatic conversion of the substrate to the product of the enzymatic reaction;
   c. comparing, the rate and/or extent of enzymatic conversion of the substrate to the product of the enzymatic reaction to a rate and/or extent of enzymatic conversion of the substrate to the product of the enzymatic reaction in a reaction mixture either excluding the sample composition or having a different amount of the sample composition; and
   d. identifying sample compositions that affect the rate and/or extent of enzymatic conversion of the substrate to the product of the enzymatic reaction.
13. The method of clause 12, wherein the nucleoside phosphate kinase is a thymidylate kinase and the shift in the peak of a thymine H6 proton is measured.
14. The method of clause 12, wherein the thymidylate kinase is a Mycobacterium tuberculosis, Plasmodium falciparum, Pseudomonas aeruginosa, Candida albicans, Escherichia coli, or Trypanosoma brucei brucei thymidylate kinase.
15. The method of clause 14, wherein the rate and/or extent of enzymatic conversion of the substrate to the product of the enzymatic reaction for the Mycobacterium tuberculosis, Plasmodium falciparum, Pseudomonas aeruginosa, Candida albicans, Escherichia coli, or Trypanosoma brucei brucei thymidylate kinase is compared to a rate and/or extent of enzymatic conversion of the substrate to the product of the enzymatic reaction for human thymidylate kinase.
16. The method of any one of clauses 12-15, wherein the nucleoside phosphate kinase is an adenylate kinase.

Having described this invention, it will be understood to those of ordinary skill in the art that the same can be performed within a wide and equivalent range of conditions, formulations and other parameters without affecting the scope of the invention or any embodiment thereof.

We claim:

1. A method of preparing an enzyme, comprising:
a. culturing a cell comprising an exogenous gene for expressing an enzyme in cell culture medium under conditions suitable for expression of the exogenous gene in the cell;
b. isolating the enzyme from the cell by lysing the cell in a lysis solution to produce a lysate, binding the enzyme in the lysate to a selectivity component specific to the enzyme that is optionally bound to a surface, and separating the bound enzyme from the lysate, wherein the enzyme is bound to the binding reagent in the presence of a substrate of the enzyme.

2. The method of claim 1, wherein the lysis solution comprises the substrate.

3. The method of claim 1, wherein the enzyme is bound to the selectivity component in a molar excess of the substrate.

4. The method of claim 1, further comprising, after isolating the enzyme from the cell, dissociating the substrate from the enzyme.

5. The method of claim 2, wherein the substrate is dissociated from the enzyme by contacting the enzyme with non-denaturing amounts of a protein denaturant.

6. The method of claim 3, wherein the enzyme is contacted with from 0.5 M to 3 M urea, or equivalent amounts of guanidinium HCl, to dissociate the substrate from the enzyme.

7. The method of claim 5, wherein the enzyme is a Homo sapiens, Mycobacterium tuberculosis, Plasmodium falciparum, Pseudomonas aeruginosa, Candida albicans, Escherichia coli, or Trypanosoma brucei brucei thymidylate kinase.

8. A method of monitoring an enzymatic reaction of a nucleoside phosphate kinase, comprising:
a. conducting a reaction of the nucleoside phosphate kinase in a reaction mixture comprising a pyrimidine substrate of the nucleoside phosphate kinase having a pyrimidine H6 proton or a purine substrate of the nucleoside phosphate kinase having a purine H8 proton; and
b. conducting a nuclear magnetic resonance (NMR) assay on the reaction mixture during the course of the reaction to identify or determine the presence of a shift in a peak of the spectrum corresponding to the pyrimidine H6 proton or purine H8 proton,
wherein a shift in the peak corresponding to the pyrimidine H6 proton or purine H8 proton indicates enzymatic conversion of the substrate to the product of the enzymatic reaction.

9. The method of claim 8, wherein the nucleoside phosphate kinase is a thymidylate kinase and the shift in the peak of a thymine H6 proton is measured.

10. The method of claim 8, comprising determining the rate of the enzymatic conversion of the substrate to the product of the enzymatic reaction or extent of the enzymatic conversion of the substrate to the product of the enzymatic reaction by comparing the spectrum peak or peaks corresponding to the pyrimidine H6 proton or purine H8 proton at two time points during which the enzymatic reaction is conducted.

11. The method of claim 10, wherein the nucleoside phosphate kinase is an adenylate kinase.

12. A method determining the effect of a composition on an enzymatic reaction of a nucleoside phosphate kinase, comprising:
a. conducting a reaction of the nucleoside phosphate kinase in a reaction mixture comprising a pyrimidine substrate of the nucleoside phosphate kinase having a pyrimidine H6 proton or a purine substrate of the nucleoside phosphate kinase having a purine H8 proton and a sample composition;
b. conducting a nuclear magnetic resonance (NMR) assay on the reaction mixture during the course of the reaction to identify or determine the presence of a shift in a peak of the spectrum corresponding to the pyrimidine H6 proton or the purine H8 proton, wherein a shift in the peak corresponding to the pyrimidine H6 proton or the purine H8 proton indicates enzymatic conversion of the substrate to the product of the enzymatic reaction;
c. comparing, the rate and/or extent of enzymatic conversion of the substrate to the product of the enzymatic reaction to a rate and/or extent of enzymatic conversion of the substrate to the product of the enzymatic reaction in a reaction mixture either excluding the sample composition or having a different amount of the sample composition; and
d. identifying sample compositions that affect the rate and/or extent of enzymatic conversion of the substrate to the product of the enzymatic reaction.

13. The method of claim 12, wherein the nucleoside phosphate kinase is a thymidylate kinase and the shift in the peak of a thymine H6 proton is measured.

14. The method of claim 12, wherein the thymidylate kinase is a Mycobacterium tuberculosis, Plasmodium falciparum, Pseudomonas aeruginosa, Candida albicans, Escherichia coli, or Trypanosoma brucei brucei thymidylate kinase.

15. The method of claim 14, wherein the rate and/or extent of enzymatic conversion of the substrate to the product of the enzymatic reaction for the Mycobacterium tuberculosis, Plasmodium falciparum, Pseudomonas aeruginosa, Candida albicans, Escherichia coli, or Trypanosoma brucei brucei thymidylate kinase is compared to a rate and/or extent of enzymatic conversion of the substrate to the product of the enzymatic reaction for human thymidylate kinase.

16. The method of claim 12, wherein the nucleoside phosphate kinase is an adenylate kinase.
